(19) United States
(12) Patent Application Publication
(10) Pub. No.: US 2022/0015474 A1
(43) Pub. Date: Jan. 20, 2022

(54) ANTI-BACTERIAL AND ANTI-VIRAL, SMART FACEMASK

(71) Applicant: City University of Hong Kong, Kowloon (HK)

(72) Inventors: Ruquan YE, Kowloon (HK); Libei HUANG, Kowloon (HK)

(21) Appl. No.: 17/378,363

(22) Filed: Jul. 16, 2021

Related U.S. Application Data
(60) Provisional application No. 63/052,494, filed on Jul. 16, 2020.

(51) Int. Cl. A41D 13/11; A61L 9/20; A61L 9/22

(52) U.S. Cl. CPC A41D 13/1192; A41D 13/1146; A61L 9/20; A41D 2500/50; A41D 2500/20; A41D 2500/40; A61L 9/22

(57) ABSTRACT

A facemask having a graphene layer providing antibacterial and antiviral properties. The facemask is also able to generate electricity from the wearer's breath. The graphene is a three dimensional graphene produced in situ the facemask by burning a suitable material in the facemask with a laser, i.e. laser induced graphene (LIG). Typically, the graphene comprises hydrophilic graphene, in such a way that the graphene is more hydrophilic towards one side of the substrate and less hydrophilic towards the other side of the substrate, which provides the possibility of generating an electrical potential difference using human breath.

### CROSS-REFERENCE TO RELATED APPLICATIONS

The present invention claims priority to U.S. Provisional Application No. 63/052,494 filed with the United States Patent and Trademark Office on Jul. 16, 2020, and entitled, “ANTIBACTERIAL ARTICLE AND USE OF THE SAME”, which is incorporated herein by reference in their entirety for all purposes.

### FIELD OF INVENTION

The present invention relates to facemasks. In particular, the invention relates to anti-bacterial and anti-viral facemasks.

### BACKGROUND OF THE INVENTION

It has become more commonplace to be wearing facemasks nowadays, even in daily life. It used to be that facemasks are worn in laboratories or where the wearer wants to prevent inhalation of smells and particles. In some countries it has become a matter of social etiquette to be wearing facemasks when one has a cold to avoid passing the cold to other people. During an outbreak of germs or viruses, such as Ebola and COVID-19, it has become necessary to prevent or to retard a pandemic by wearing of facemasks. When the wearer eats or drinks, he has to remove his facemasks. Preferably, the wearer changes a fresh facemask when he has finished his meal. However, if there is an outbreak of a pandemic and facemasks becomes part of daily attire, the expense of changing a few facemasks throughout the day becomes economically and environmentally unviable. Hence, many people would resort to re-wearing a used facemask. Some types of facemask are made of cloth meant to be washed and reused, and some other types are made of paper or polymer and are intended to be single, one-time use. Where facemasks are regularly worn, it becomes a hassle to change a fresh one every time. However, it is unhygienic to re-use facemasks.

However, there is no way the wearer can wash a disposable facemask without damaging the mask structure, as a disposable facemask is not intended to be re-useable. He cannot spray the facemask with disinfectant after taking it off to have a meal, and wearing the facemask again after the meal. Wet facemasks cannot be worn comfortably. Even so, liquid disinfectant is only effective against a spectrum of microbes and is still likely to promote the growth of other types of microbes. The wearer also cannot lay the facemask out to sun while he takes a meal, and guard the masks from birds and people while sunning the facemask.

Accordingly, it is desirable to propose devices and or methods that cannot make facemasks more hygienic and easier to reuse.

### SUMMARY OF THE INVENTION

In the first aspect, the invention proposes a facemask comprising a substrate having an inner surface for being worn over the mouth and nose of a person; the substrate having an outer surface for facing away from the mouth and nose of the person; and the inner surface comprises three dimensional graphene.

Therefore, the invention provides the possibility of a facemask, or a surgical mask, that has properties of graphene. In particular, the facemask has antibacterial and antiviral properties. In example embodiment, the antibacterial article is a surgical mask.

Majority of bacteria on commercial activated carbon mask and surgical mask may remain alive even after 8 hours. By using graphene, the inhibition rate improves to about 81%. If the facemask is brought into the sun, under the photothermal effect induced in graphene, 99.998% bacterial killing efficiency could be attained within 10 minutes.

Preferably, the three dimensional comprises laser induced graphene. Use of laser to induce graphene provides the possibility of inducing a graphene layer on a facemask made in the conventional way. That is, a normally manufactured facemask made of a suitable material can be lased to create graphene in situ on the facemask. In contrast, making a graphene layer separate to be integrated into a facemask disrupts existing facemask manufacturing process and logistics.

Preferably, the three dimensional comprises hydrophilic graphene.

More preferably, the graphene is more hydrophilic towards one side of the substrate and less hydrophilic towards the other side of the substrate. This creates a graduated patterning of the graphene. By patterning the graphene materials, moisture-induced electricity can be generated when people inhale or exhale. This moisture-induced electricity can be used to power low-power electronics or track the masks conditions. In other words, the invention provides the possibility of a hygroelectric graphene facemask. Furthermore, the induced voltage might also improve the adsorption/filtering efficiency.

Furthermore, the invention therefore provides the possibility of a facemask which is able to contain and kill infectious species, and also capable of providing an indication of the condition of the mask, such as if there are too much bacteria accumulated onto the facemask. In addition, the invention also provides the possibility of a facemask that can be a power source for some electronic devices.

Information on the accumulation of bacteria on the facemask is important for doctors and nurses who are in close contact with patients. Both medical personnel and the patient can be monitored for the amount of bacteria they exhaled.

Preferably, a diode that is arranged across the graphene such that electrical potential difference generated in the graphene is able to light the diode.

Alternatively, electrochromic material that is arranged across the graphene such that electrical potential difference generated in the graphene is able to change the electrochromic material chromatically.

Therefore, the invention provide the possibility of a crude pre-diagnostic tool in the form of a facemask, in that the faster a facemask fills up with bacteria, the greater the likelihood the wearer needs to be examined by a doctor for a possible infection.

Preferably, the substrate comprises any one of the following: polyimide, paper, polyethersulfone, polysulfone, melt-blown fabrics, woven fabrics and felted-fabrics.

In the first aspect, the invention proposes a method of functionalizing a carbonaceous material comprising the steps of: providing a carbonaceous material; a first stage of applying laser onto the carbonaceous material to produce a layer of three dimensional graphene in the presence of an inert atmosphere; a second stage of applying laser onto the three dimensional graphene in the presence of air; wherein the second stage comprises applying laser to a first part of the three dimensional graphene in such a manner that provides functionalization of the first part with polar groups; and applying laser to a second part of the three dimensional graphene in such a manner that provides different extent of functionalization of the second part with polar groups.

Preferably, functionalization of the first part with polar groups comprises applying a number of laser pulses to the first part; and functionalization of the second part with polar groups comprises applying a different number of laser pulses to the second part.

Preferably, functionalization of the first part with polar groups comprises applying one laser intensity to the first part; and functionalization of the second part with polar groups comprises applying a different laser intensity to the second part.

Preferably, functionalization of the first part with polar groups comprises applying one laser intensity to the first part; and functionalization of the second part with polar groups comprises applying a different laser intensity to the second part.

Therefore, in a further aspect, the invention proposes a filter made of graphene material, which can be either a single filtering layer, or on the surface of substrates such as melt-blown fabrics or other cloth. The graphene is patterned with gradient oxidation. The gradient oxidation forms the hygroelectric generator, which harvests energy from human breath.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 illustrates a facemask that is a possible embodiment of the invention;

FIG. 2 shows the structure of graphene that can be used in the embodiment of FIG. 1.

FIG. 3 shows how the inner side of the facemask of FIG. 1 is treated by laser to convert the substrate surface into graphene;

FIG. 4 shows the molecular structure of polyimide that may be used in the embodiment of FIG. 1;

FIG. 5 shows darkened layers inscribed onto a piece of polyimide, similar to polyimide used in the embodiment of FIG. 1;

FIG. 6 shows the molecular structure of PES that may be used in the embodiment of FIG. 1;

FIG. 7 shows Raman spectra of graphene that may be found in the embodiment of FIG. 1;

FIG. 8 shows a beam of CO2 laser applied onto the embodiment of FIG. 1 in the presence of an inert gas such as nitrogen or argon.

FIG. 9A shows the laser applied onto the embodiment of FIG. 1 in the presence of air;

FIG. 9B shows the different in surface contact angle of different laser induced graphenes (LIGO-), one having been prepared in air and the other in an nitrogen atmosphere;

FIG. 10 shows graphene that is alternative to the graphene shown in FIG. 8 and FIG. 9A;

FIG. 11 shows the scanning electron microscopy (SEM) image of the graphene that may be produced on the embodiment of FIG. 1;

FIG. 12 shows plots of temperature of the surface of the graphene that can be provided in the embodiment of FIG. 1 against time;

FIG. 13 is an optical image on the left side and an IR image on the right side, of a piece of graphene that can be provided in the embodiment of FIG. 1;

FIG. 14 illustrates how a graphene facemask of FIG. 1 can be further treated by laser;

FIG. 15 is a schematic illustration of further details of the process shown in FIG. 14;

FIG. 16 illustrates how three dimensional graphene can produce an electrical potential difference when met with a passing cloud of moist air;

FIG. 17 illustrates the working mechanism of the hygroelectric graphene obtained by the process of FIG. 14;

FIG. 18 illustrates the application of the embodiment obtained by the method of FIG. 14;

FIG. 19 demonstrates the correlation between the graphene of FIG. 14 and functionalizing pulses;

FIG. 20 show the effects in the embodiment of FIG. 14;

FIG. 21 illustrates electrical potential difference that may be seen in the embodiment of FIG. 14;

FIG. 22 illustrates the embodiment of FIG. 14 in use;

FIG. 23 is a plot of current that may be observed when the embodiment of FIG. 14 is use;

FIG. 24A to FIG. 24F were SEM images of three kinds of mask materials, showing different viability of E. coli;

FIG. 25 shows the bacteria colony count on difference facemask materials;

FIG. 26A to FIG. 26D shows the morphological difference of E. coli on hydrophilic LIG and hydrophobic LIG;

FIG. 27 shows the E. coli viability on two kinds of graphene;

FIG. 28 shows the UV-Visible-Near Infrared spectrum of LIG and its precursor, which indicates that LIG can effectively absorb the energy from 250 nm to 2750 nm;

FIG. 29 summarizes the change of temperature with time of LIG, ACF and MBF under 1 Sun irradiation;

FIG. 30 is a plot of the antibacterial efficiency of different facemask materials; and

FIG. 31 shows number of bacteria colonies that maybe formed on different facemask materials.

### DETAILED DESCRIPTION OF SPECIFIC EMBODIMENTS

FIG. 1 illustrates a single use facemask 101. The facemask is generally a flat, flexible substrate 103 that is made of cloth, paper, polymer or a mixture of these materials. The shape of the mask is usually rectangular. The ends of the rectangle are provided loops 105 for hooking onto the ears of the wearer.

The flat substrate has two sides, one to be placed against the mouth and nose of the wearer which will be called the inner side here, while the other faces away from the wearer (marked with arrow A) which will be called the outer side here. The inner side tends to accumulate large amounts of viruses and bacteria, as the wearer breathes into the facemask. To kill bacteria and viruses on the inner side, the inner side is provided with a layer of graphene.

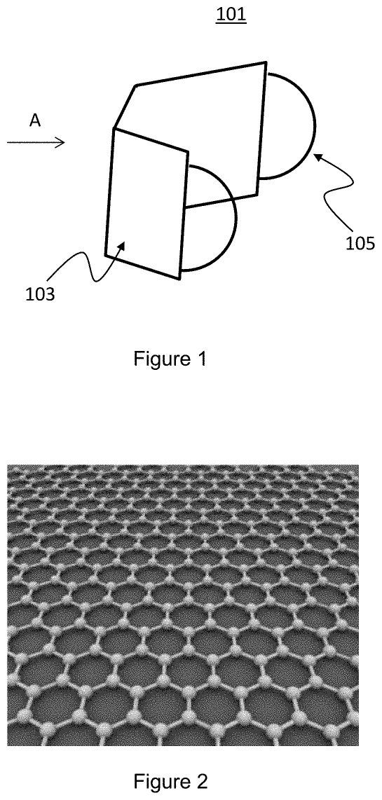

FIG. 2 shows the structure of graphene (image obtained from Wikipedia.org). Typically, graphene is an allotrope of carbon consisting of a single layer of atoms arranged in a two-dimensional honeycomb lattice. In other words, graphene is a very simple structure of graphite, as graphite is simply many stacked layers of slidable graphene. As the skilled man knows, graphene has excellent electrical, mechanical, and thermal properties.

In some embodiments, the inner side of the facemask is inserted with a layer of fabric comprising a graphene surface, as additional lining to an existing facemask. In the preferred embodiment, however, a graphene layer is provided by converting the surface of inner side of the facemask into graphene.

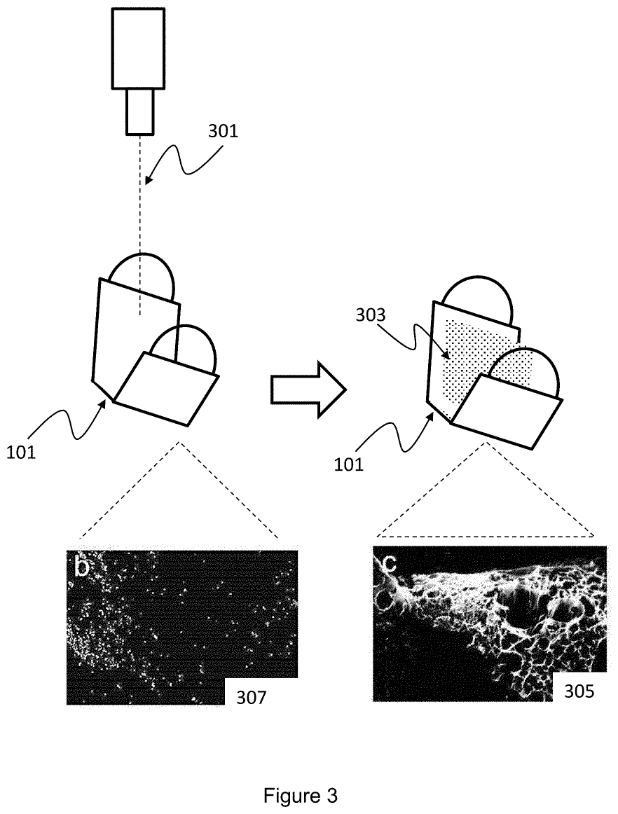

FIG. 3 shows how the inner side is treated by laser 301 to convert the substrate surface into graphene. The substrate is a carbon-based material, and lasing the surface of the substrate carbonises the surface the substrate. In the process, however, with a suitable laser frequency and intensity, the surface of the substrate is not completely carbonised, but becomes a three dimensional porous structure of randomly connected graphene. Typically, the laser is CO₂ laser. This graphene layer 303 looks like a blackened part of the substrate 305. In comparison, the untreated substrate does not show any three dimensional porous structure, and only shows a relatively flat structure 307.

The substrate can be made of any carbon-based materials, including both biogenic and synthetic polymers. Use of biomaterials lends an advantage of a ready supply of raw material, which relieves supply stress and environmental pressure if there is any pandemic outbreak which causes a sudden spike in mask demand and supply.

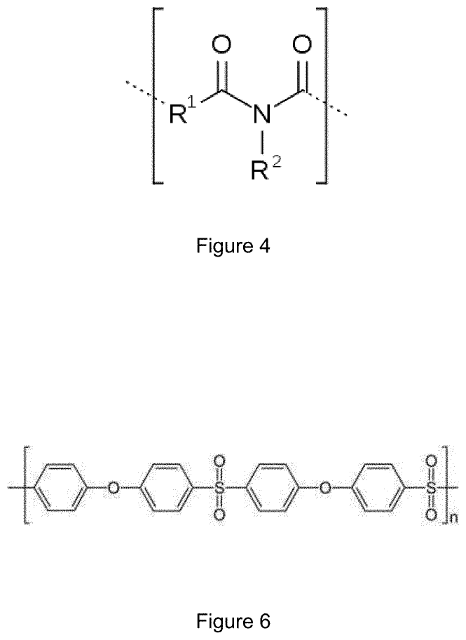

Preferably, however, the material in the substrate for producing graphene is a polyimide film. Polyimides are polymeric plastic material with high thermal stability, and therefore undergo the lasing without burning up too much, and thereby encouraging the formation of the porous, laser-induced graphene structure. FIG. 4 shows the molecular structure of polyimide, where R1 and R2 are organic chains of any possible lengths.

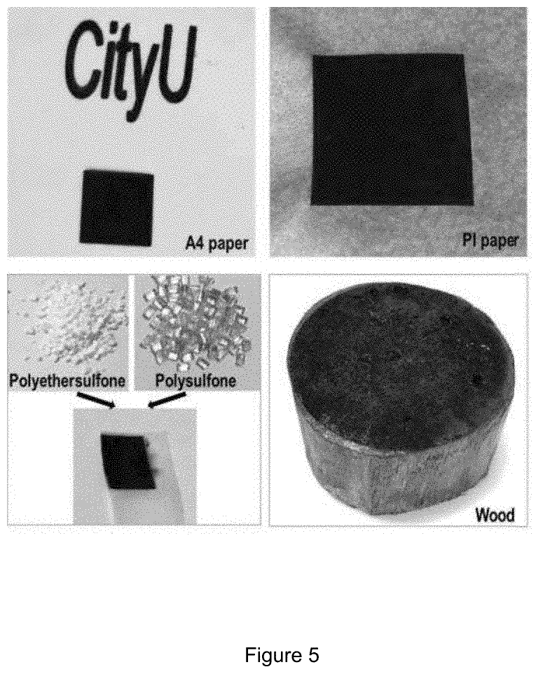

Other materials can also been demonstrably lased to convert the materials into graphene. FIG. 5 shows darkened layers inscribed onto a piece of polyimide using laser, the top left shows “CityU” written on pulp-based writing paper using laser, the top right shows a dark square created on paper that contains polyimide by laser, the bottom left shows a dark square created on paper that contains polysulfone and polyethersulfone by laser, and the bottom right drawings shows a piece of wood which has been treated with laser to have a graphene surface.

Polysulfones (PES) is a family of high performance thermoplastics. These polymers are known for their toughness and stability at high temperatures, and therefore very suitable for being subjected to lasing. FIG. 6 shows the molecular structure of PES.

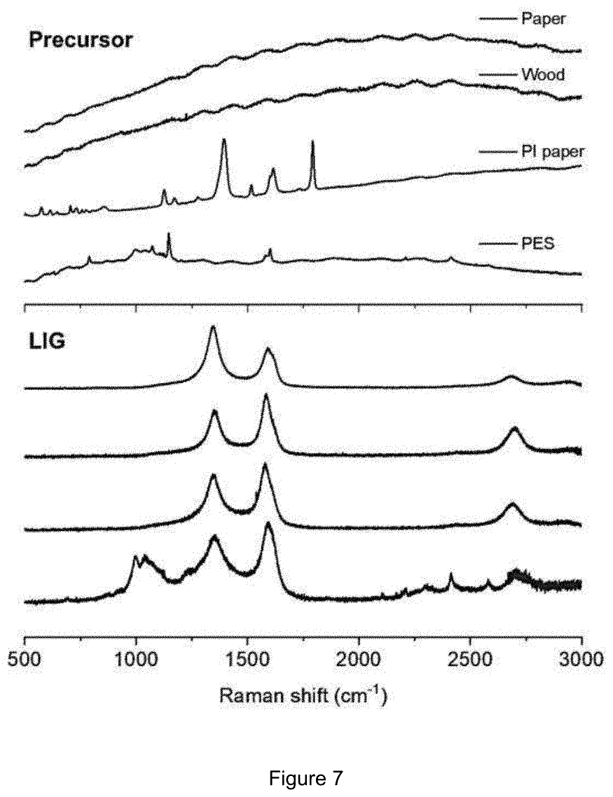

FIG. 7 shows Raman spectra showing that on being lased, paper, wood, polyimide paper (paper containing polyimide) and PES (polyethersulfone) all show the three characteristic peaks of graphene materials (G peak at about 1350 cm⁻², D peak at about 1590 cm⁻² and 2D peak at about 2700 cm⁻²). This proves the conversion of the materials into graphene.

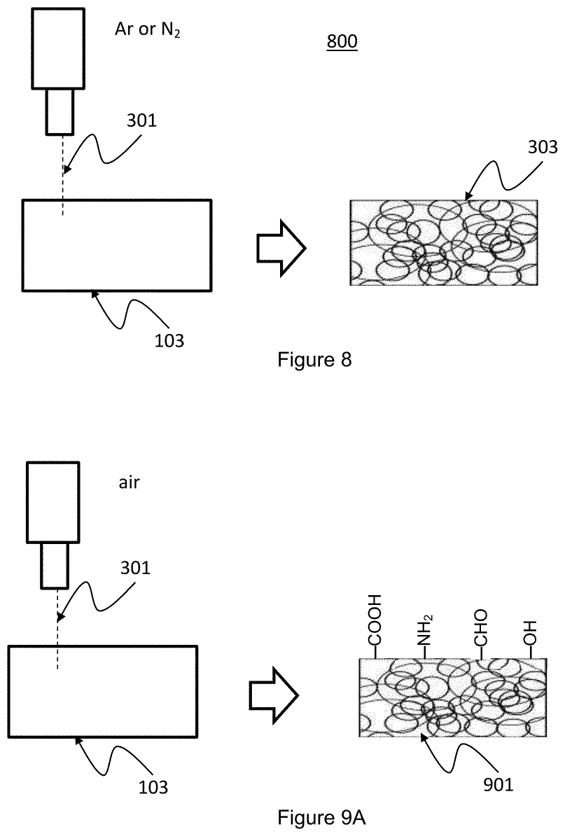

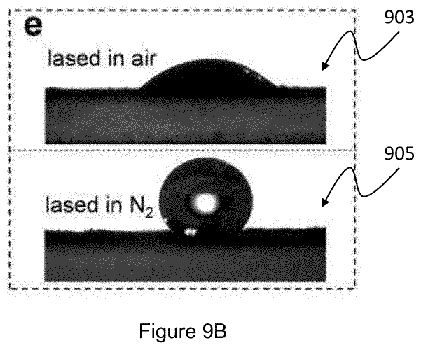

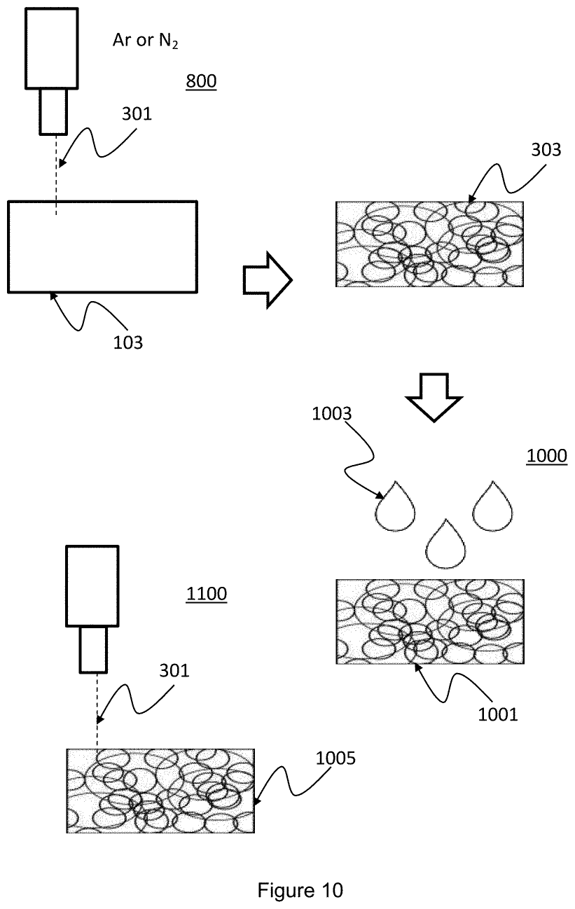

FIG. 8, FIG. 9A and FIG. 10 illustrate how two different types of graphene layers may be produced onto the facemask. FIG. 8 shows a beam of CO2 laser applied onto the substrate in the presence of an inert gas such as nitrogen or argon, at step 800. In this case, the graphene 303 produced is basically carbon, and has a strong hydrophobic character, called Laser Induced Graphene (LIG) in the rest of this description.

FIG. 9A shows the laser applied onto the substrate in the presence of air. In this case, the graphene produced is randomly functionalized with polar molecular groups, as oxygen, carbon dioxide and moisture in the air is caught in the lasing process and chemically attach to the graphene surface. This gives a strong hydrophilic character to the graphene, called Hydrophilic Laser Induced Graphene (HLIG) 901 in the rest of this description.

FIG. 9B shows that, when lased in air, the LIG shows a contact angle of 20°, at 903. An angle of about 140° was obtained when lased in an inert atmosphere, at 905, demonstrating the hydrophobicity of HLIG.

FIG. 10 shows yet how a further type of graphene layer that is embedded with silver nano-particles 1001. At first, the LIG 303 is produced on the substrate 103 by the same way as shown in FIG. 8. That is, one lase was applied to create a LIG film, at step 800. The laser power, speed, pulses/dot and line spacing were set as 1.8 W, 1000 mm/s, 5 and 0.03 mm, respectively. The laser mode was set as vector mode 10 g/mL AgNO₃ solution 1003 was then loaded on the obtained LIG film drop by drop, with a loading amount of 1 mg/cm², and then dried it in room temperature, at step 1000. Finally, a second lase process is applied, at step 1100, to the LIG surface, with the same laser conditions as the first lase was applied, and a silver nano-particle laser induced graphene (Ag NPs/LIG) composite 1005 is produced.

Graphene surfaces as shown in FIG. 8, FIG. 9A and FIG. 10 have found to inhibit the survival of bacteria. The inhibition rate improves by as much as 81% compared to inner surface of facemasks. Typically, the inner surface of facemasks is made of a polymeric material such as polypropylene, which is made in a process called melt blown extrusion for creating non-woven fabric. The melt blow extrusion process produce fabric that is constructed of generally smaller, more delicate fibres that is generally considered washable or reusable.

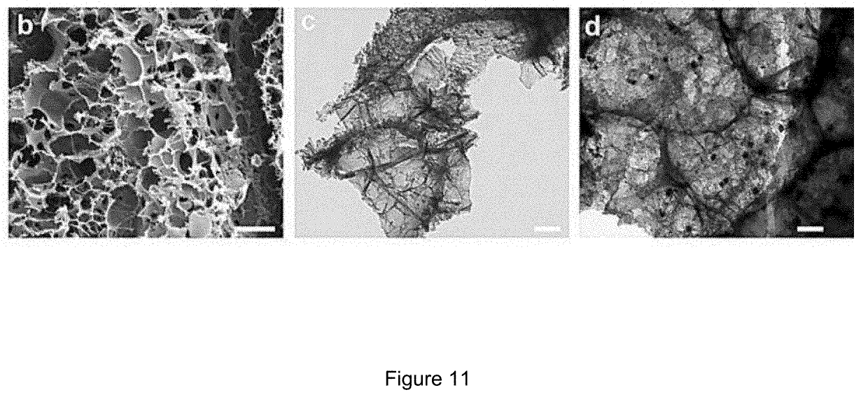

FIG. 11 shows the scanning electron microscopy (SEM) image of the graphene that may be produced on facemasks. The leftmost image in FIG. 11 illustrates the LIG, with pore sizes of several hundreds of nanometres clearly visible in the highly porous three dimensional graphene structures. The transmission electron microscopy (TEM) images in the centre image, and the rightmost image show the few-layered graphene structure and uniform distribution of spherical Ag nano-particles of a size of tens of nanometres.

When a bacteria or a virus contacts graphene, the bacteria or virus can be killed. There are various possible mechanisms of graphene antibacterial properties, such as oxidative stress, membrane stress, and electron transfer that act on the membrane of bacteria and viruses. For example, graphene can physically damage the bacterial membranes by direct contact. Further elaboration of the specific mechanisms is not necessary here.

Accordingly, the embodiment provides an anti-bacterial and anti-viral facemask. This allows the wearer to re-use the facemask in normal daily activities to a reasonable extent without concern that the facemask has accumulated too much bacteria or virus.

Furthermore, the anti-bacterial and anti-viral effects are found to be improved by photothermal effects. Photothermal aided graphene is able to kill 99.998% of bacterial on the graphene surface within 10 minutes under 1 Sun (1 kW/m2) irradiation. Hence, when the wearer steps into the sun, the anti-bacterial and anti-viral effects of the facemask are even more pronounced.

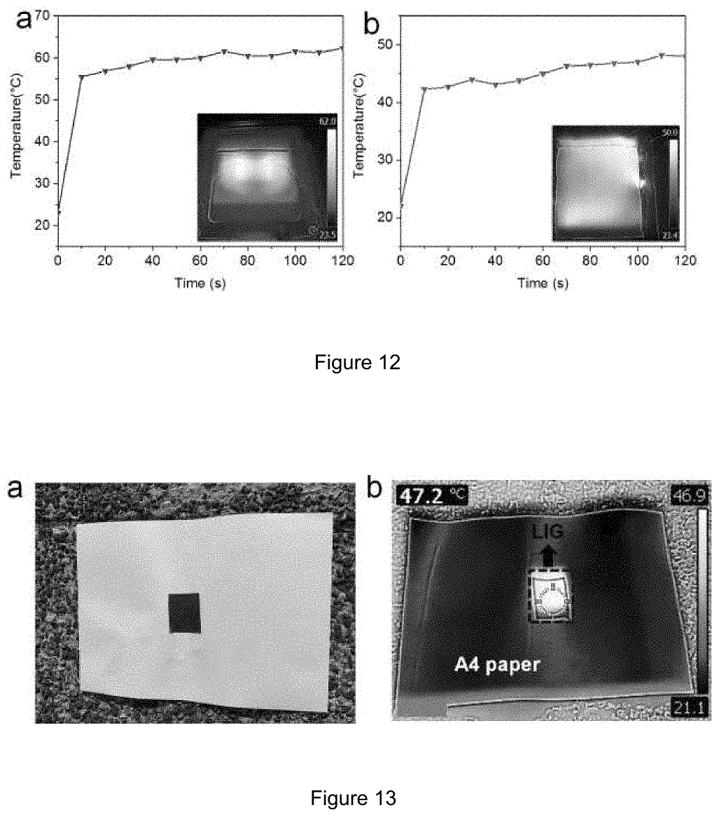

FIG. 12 shows plots of temperature of the surface of the graphene against time. The rightmost picture shows how a small piece of the graphene, a piece of 4×4 cm² LIG under 1 kW/m² simulated Xenon sunlight. The leftmost shows how a small piece of graphene, a piece of 10×10 cm² LIG, under direct current voltage of 7.5 volts. Under both the influence of sunlight and electricity, the graphene generates surficial temperature of 50 degrees Celsius to 60 degrees Celsius.

FIG. 13 is an optical image on the left side and an IR image on the right side, of a piece of LIG. The ambient temperature is 8-14° C., relative humidity is 25%, wind speed is 18 km/h. However, the surface temperature of LIG increases to about 47° C. when exposed to sunlight outside despite the mild ambient conditions, which indicates that the virus on LIG can be inactivated in mild conditions.

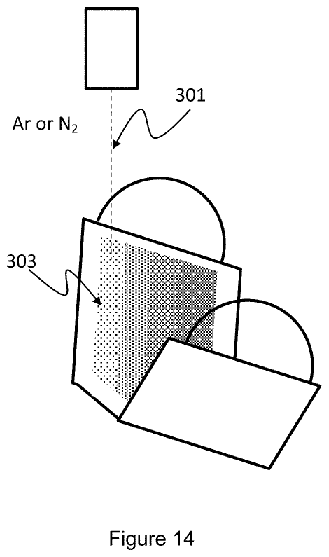

FIG. 14 illustrates how the graphene 303 in the facemask 101 can be further treated by another bout of the same type of laser 301 as that described as used in FIG. 8, but now in air. However, the earlier procedure in step 800, the laser 301 is applied across the substrate evenly, such that the graphene 303 is created in situ and evenly over the substrate 103. In this later step, the laser 301 is applied with variation, steadily changing as the laser is moved linearly across the layer of graphene. The variation can be in the number of pulses of laser on every next point, or ‘dot,’ as the laser moves across the layer of graphene. Alternatively, the variation can be in intensity of laser on every next point, or ‘dot,’ as the laser moves across the layer of graphene.

Eventually, the variation in the second lase applied across the layer of graphene functionalises the surface of the graphene to different extents, respectively. It has been proposed that the second lase in the presence of air creates functional or hydrophilic groups on the graphene surface. The surface of graphene towards one side of the facemask is more functionalised than the surface of graphene towards the other side of the facemask. The functionalization changes gradually across the facemask, creating a gradient of more functionalization from the one side to less functionalization the other side.

The different number of pulses renders the LIG's surface properties with a proportional degree of oxidation, hydrophilicity and conductivity. As shown in FIG. 14, the layers of graphene can be seen to be darker towards one side and light towards another side, illustrating that the surface of the LIG gradually changes from mildly oxidized to highly oxidized, going from the lower number of pulses to a high number of pulses, respectively.

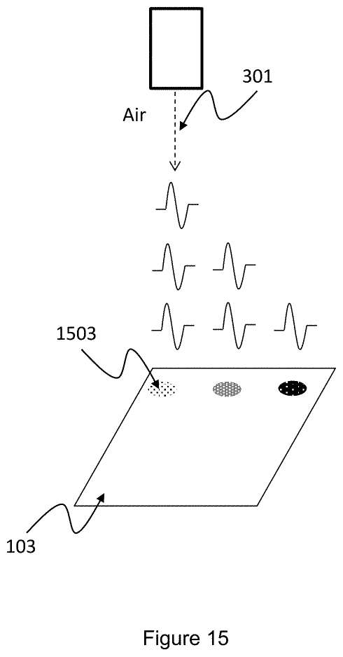

In FIG. 15, a schematic illustration shows three pulses used to create one dot, two pulses to create another dot, and a pulse to create yet another dot. The dots 1503 in FIG. 15 are produced by lasing across the breadth of the inner surface of the substrate 103 in the facemask.

Graphene is hygroscopic and attracts moisture. The gradient of oxidation, hydrophilicity and conductivity creates a corresponding gradient distribution of protons when humid air passes through the graphene. This creates a moisture-induced potential difference across the graphene. Thus, the layer of graphene is a hygroelectric generator powered by human breath, termed “hygroelectric LIG” herein.

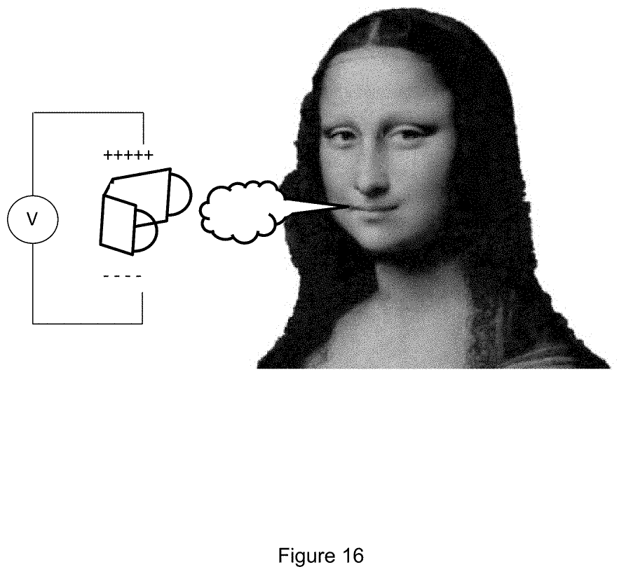

As illustrated in FIG. 16, the gradually oxidized layer of three dimensional graphene can produce an electrical potential difference when met with a passing cloud of moist air. Hence, it is possible to generate electricity on the facemask.

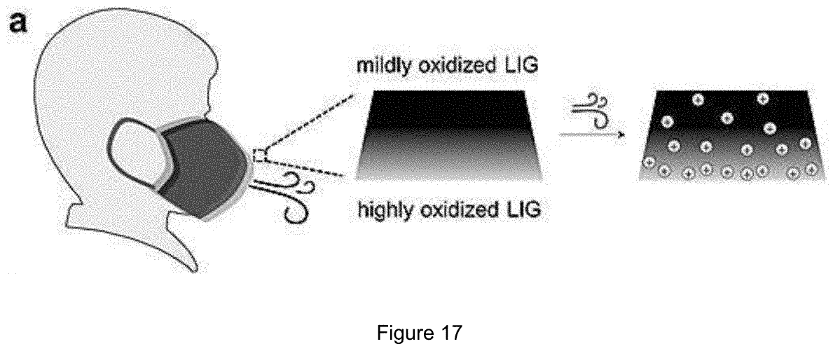

FIG. 17 illustrates the working mechanism of the hygroelectric LIG. Electricity can be generated in the graphene because a gradient distribution of protons can be generated when humid air passes through a hygroscopic surface. Therefore, when the facemask wearer exhales, the hydrophilic part of LIG adsorbs moisture and a gradient of protons distribution forms due to the oxidation gradient across the graphene. The gradient protons concentration induces an internal electric field and creates free electron movement of external circuit. When the diffusion (induced by gradient concentration) and drifted effect (forced by internal electric field) of protons reached a dynamic balance, the induced potential difference reaches a zenith. When the wearer inhales, moisture is removed from the graphene surface. As the graphene dehydrates, the protons recombine with the negatively charged groups, thereby relaxing the induced potential to its initial state. The difference in humidity between inhalation and exhalation is typically about 30%. Therefore, a voltage arises and descends when the wearer inhales and exhales

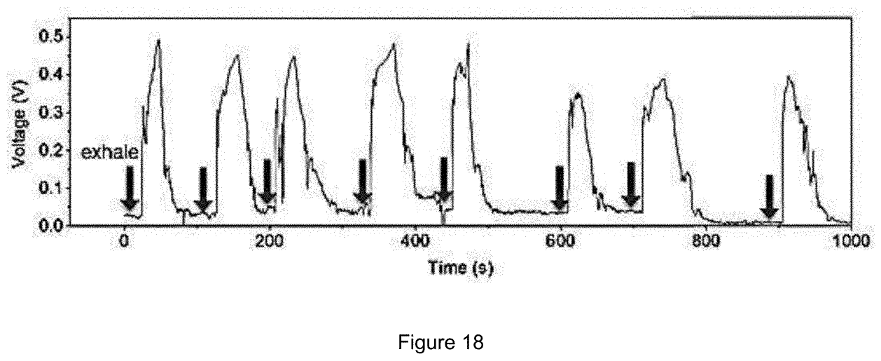

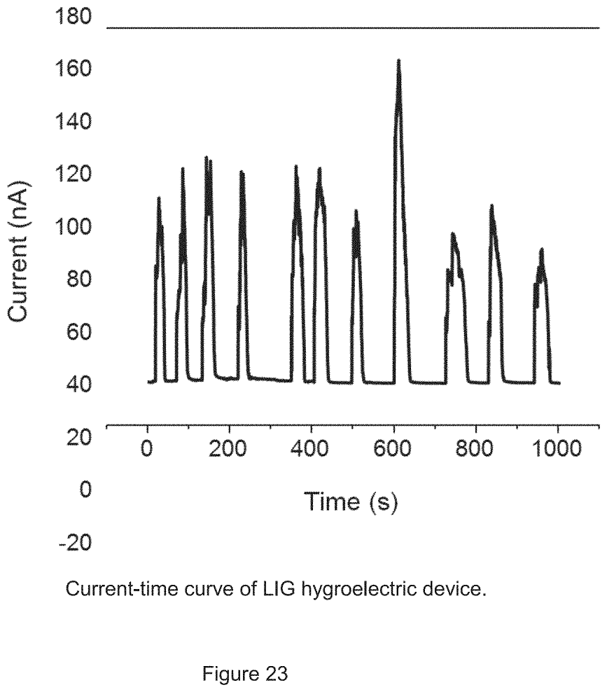

As the voltage-time curve shown in FIG. 18, when individuals breathes out, the induced voltage ramps up to 300 mV to 600 mV within 3 seconds, and it takes 50 seconds to 70 seconds to return to the original state. Simultaneously, therefore, a current output of 100 nA to 160 nA is attained as FIG. 23 shows. It has been found that the initial averaged voltage output of LIG device was about 0.4 V, and it hardly changed even after a cleaning process, indicating the wetting process by water does not affect the surface properties and the induced potential. Therefore, human breath can be used to create an electrical potential.

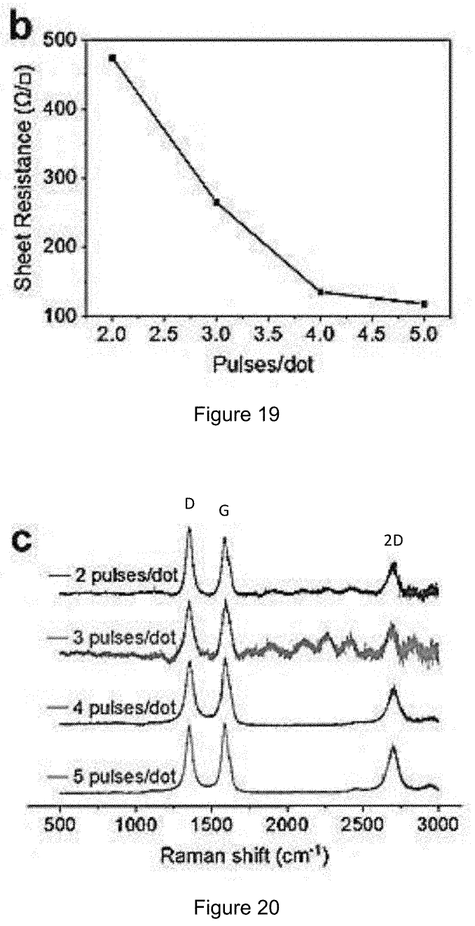

FIG. 19 demonstrates the correlation between LIG's sheet resistance and pulses per dot of laser, where the vertical axis is in ohms per metre and the horizontal axis in pulses per dot. The greater the number of pulses, the less resistance is induced in the graphene.

The Raman spectra in FIG. 20 show the effect of pulses on the graphene structure. The skilled reader would be able to see that the intensities of the D band and the G band (ID/IG) of the LIG lased with 2 pulses per dot is greatest among the Raman spectra, and has a noisy background. As the skilled reader would know, ratio of the intensity of D-Raman peak and the G-Raman peak (ID/IG) is used for characterization of carbon films, for example to estimate number and size of the sp² clusters. As the number of laser pulses per dot increases, the full width at half maximum (FWHM) of LIG became narrower and the ID/IG decreases, indicating a graphene structure with lower defect and higher crystallinity.

Besides using different number of pulses to create different oxidised extent of each dot, spacing between each applied line of laser, change in speed of lasing each line on the graphene can also be used to achieve the same effect.

A small light-emitting diodes and liquid-crystal display can be connected to the facemask by simply connecting the LIG hygroelectric generators in serials or parallel, to generate light or display from the electricity. This can be used to allow one to see if the wearer is breathing, and has use in medical monitoring of people, or safety monitoring of people such as miners working in dark tunnels.

Alternatively, a small colour strip or foil that changes colour when an electrical potential difference is applied across the strip can be woven into the facemask. An example of such technology is electrochromic materials which will change, evoke or bleach their colour in response to a small amount of electricity (see for example, https:www.americanscientist.org/articles/switching-colors-with-elecitricity). These materials can be made of metal oxides, conjugated conducting polymers, viologens, metal coordination complexes, prussian blue.

In a further embodiment, the LIG hygroelectric generator can be used to power a “smart” mask that is capable of reporting the condition of the mask. Since the moisture-induced electricity is established from the gradient hydration ability of LIG surface, the accumulation of bacteria will destroy the surface gradient, and eventually dismiss the induced potential when the load of bacteria on the graphene is high.

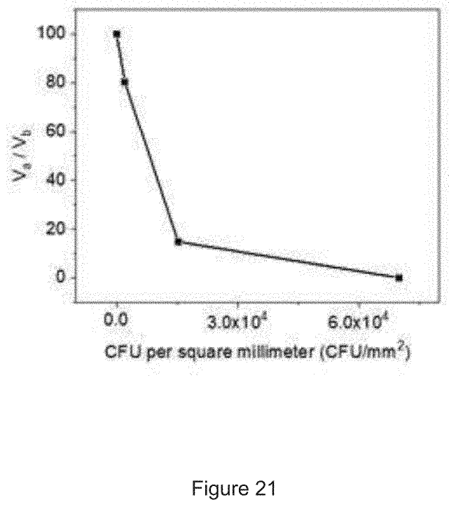

FIG. 21 illustrates how the electrical potential difference generated before and after different amounts of E. coli is adhered to the inner side of the mask.

The vertical axis shows the ratio between the voltage after bacteria adhesion to voltage before adhesion, VaNb. The Va Nb versus the amount of bacteria loading per unit area on the graphene shown in FIG. 21.

When the amount of bacteria caught on the LIG is just about 0.5×10⁴ CFU/mm², the voltage reduces to just 80% of its initial value. The induced voltage further reduces as the amount of deposited bacteria increases. Eventually, no voltage could be generated at a bacterial loading of about 7×10⁴ CFU/mm². Hence, the voltage that is induced as the facemask is used can be used to estimate the amount of bacteria loaded onto the graphene.

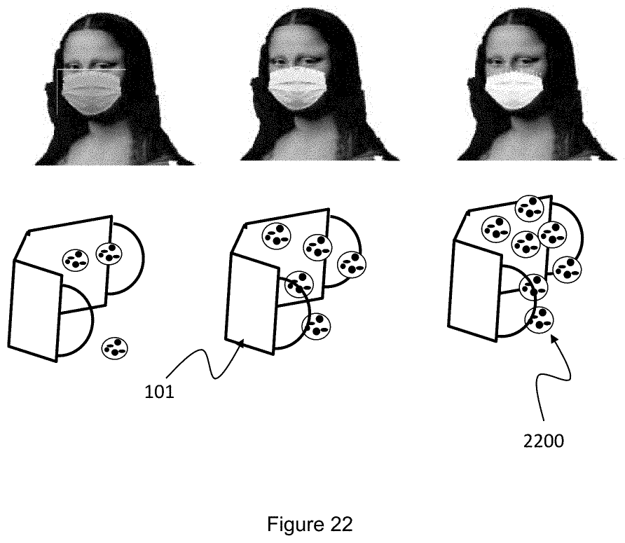

Accordingly, FIG. 22 shows an example of such an embodiment that uses the electricity that is generated by the moisture of the breath. Adhesion of bacteria on LIG changes the surface properties of LIG and reduces the moisture-induced electrical potential difference. This can be used to provide pre-diagnostic information on the build-up of bacteria on the surface of the graphene. To provide a visual indication, one or more electrochromic materials that are responsive to show different colours for different extent of electrical potential difference can be woven into the facemask. Thereby, depending on the colour of the electrochromic material on the facemask, one can tell how much bacteria had accumulated in the inner side of the mask. Thus, FIG. 22 shows a user wearing a facemask which has been installed with an electrochromic material on the outer side of the facemask. The electrochromic material is connected to the graphene on the inner side of the facemask, such that when the user breathes into the graphene, electricity is generated that can change the colour of the electrochromic material. Depending on amount of electricity generated the electrochromic material changes colour to a different extent. The actual type of colour that the electrochromic material is capable of changing into and the extent of the colour change is product specific, and is a concern in actual production but not for understanding the embodiments of the invention here.

The wearer on the leftmost in FIG. 22 has accumulated a very small amount of bacteria 2200 in the facemask 101, and there is a relatively greater colour change on the outer side of the facemask due to the high voltage induced by the wearer's breath. As more bacteria is expelled from the wearer and stuck onto the graphene, the current that may flow through the graphene is lower, as the presence of the bacteria on the graphene reduced the electrical potential difference. This creates less colour change on the facemask. When the number of bacteria has accumulated so such a huge amount, the electrical current that flows through the graphene is very much impeded. Hence, there is no colour change in the outer side of the facemask even if the wearer breathes into the facemasks. This is a crude but general pre-diagnostic tool for detecting how infectious is the wearer is or whether the facemask should be discarded. Such a self-reporting antibacterial mask improves the protection effect, especially for frontline workers at a higher risk of infection.

This provides pre-diagnostic information on the conditions of masks. Such a self-reporting antibacterial mask improves the protection effect, especially for frontline workers at a higher risk of infection.

Similar, observations can be made about viral load in the facemask. The more virus deposited onto the graphene, the less current may flow in the facemask. Therefore, the colour change in the facemask due to presence of virus is the same as that caused by presence of bacteria.

Besides facemasks, other devices or products that include a part to be worn over the breath of the wearer are within the contemplation of the embodiments, such as a motorcycle helmet.

While there has been described in the foregoing description preferred embodiments of the present invention, it will be understood by those skilled in the technology concerned that many variations or modifications in details of design, construction or operation may be made without departing from the scope of the present invention as claimed.

### Examples of Experimental Results

### Antiviral Performance

Two human coronaviruses, HCoV-OC43 and HCoV-229E, were used to evaluate the antiviral performance of three different types of LIG and melt-blown fabrics (MBF). MBF is the key filtering layer in commercial surgical masks.

Viral fluid was first separately incubated with LIG (laser induced graphene), HLIG (hydrophilic laser induced graphene), and Ag NPs/LIG (silver nano-particles laser induced graphene) and MBF (melt-blown fabric) with exposure to sunlight irradiation for 5 min, 10 min, and 15 min.

The viral fluid was then used to infect MRC-5 cells and the level of viral mRNA extracted from the infected cells was measured by “real-time polymerase chain reaction” (RT-PCR). Cell only and cell+virus were used as negative control (NC) and positive control (PC), respectively.

It has been found that cells infected with sunlight-treated virus have much lower viral RNA copies compared with the MBF group. After 10-min sunlight irradiation, the level of HCoV-OC43 RNA were decreased by 9%, 77% and 34%, and the level of HCoV-229E decreased by 29%, 30% and 68% for LIG, HLIG, and Ag NPs/LIG, respectively. Prolonged irradiation time to 15 min improved the inhibition rate of HCoV-OC43 mRNA level on LIG, HLIG, Ag NPs/LIG to 58%, 99.975% and 85.7%, and that of HCoV-229E to 99.8%, 99.5% and 75.67%, respectively. This result showed the extraordinary antiviral activity of HLIG, the viral mRNA level in MRC-5 cells was almost vanished after 15-min irradiation for both HCoV-OC43 and HCoV-229E.

The surface temperature was about 46° C. for all the LIG samples and the viral fluid was kept wet throughout the test. Yet under such mild conditions, it is sufficient to inactivate most of coronavirus. In practical use, the viral inhibition performance of HLIG could be even higher due to induced dryness.

### Stability of Antiviral Property

The stability of LIG antiviral property has been tested against HCoV-OC43 and HCoV-229E. The RT-PCR results showed that the LIG surface can maintain strong antiviral activity even after multiple uses.

On the other hand, stability of HLIG against HCoV-229E was remarkable with inhibition efficiencies of 99.97%, even after being reused three times. The results showed that the inactive effects of HLIG to coronaviruses are very stable and can be recycled for multiple uses.

The expression level of HCoV-OC43 in MRC-5 cells by immuofluorescence analysis has also been done. Compared to the positive control, almost no infected cells in LIG and HLIG could be seen. The average fluorescence of LIG and HLIG with sunlight is 13.416±1.598 and 12.14625±0.577 per cell in the testing cohorts, respectively. These counts were significantly lower than the positive control and MBF group.

Median tissue culture infective dose (TCID₅₀) assay was conducted to detect the viral titers. The results showed significant inhibition of virus infectivity by 3-5 folds after the treatment on LIGs with sunlight. Without sunlight, all the LIG are also able to show reduced infectivity, but weaker.

These data clearly show that treatment on LIG and HLIG with sunlight can effectively reduce the establishment of infection and spread of coronaviruses.

In summary, LIGs exhibits virucidal capacity, but a sharp increase to 97% and 78% against HCoV-OC43 and HCoV-229E can be attained after 15-min exposure to sunglight for HLIG. The low cost, scalable production, mild virucidal conditions, reusability and sustainability make HLIG a promising daily-use tool amid the pandemic.

### Anti-Bacterial Performance

E. coli was used to compare antibacterial performance of LIG to those of activated carbon fiber (ACF) and melt-blown fabrics (MBF). MBF is the key filtering layer in commercial activated carbon and surgical masks, respectively. The E. coli incubated for 1 h on LIG, ACF, and MBF was used as the reference, and the additional 7-h incubation was for the assessment of bacterial inhibition rate.

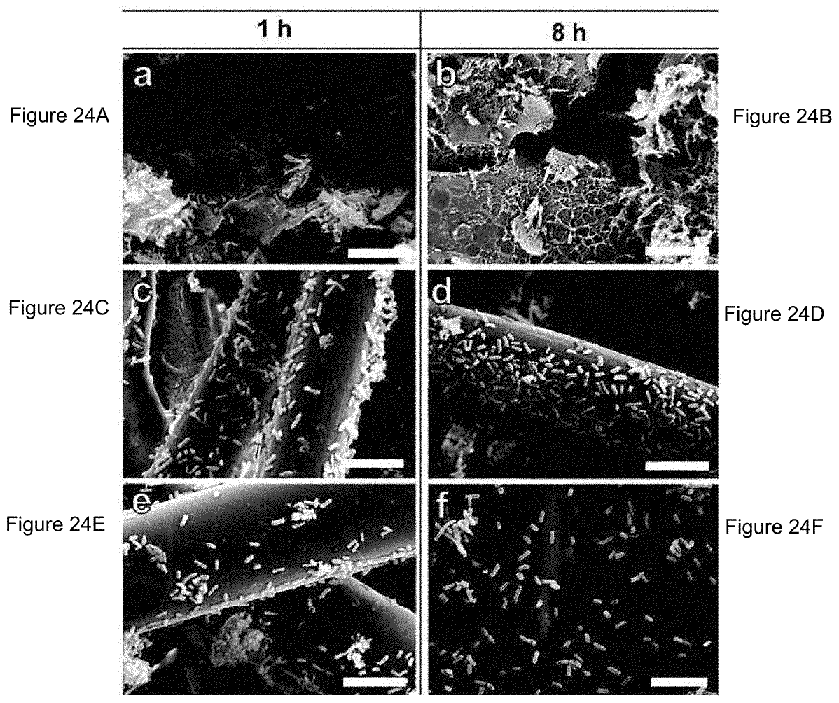

FIG. 24A to FIG. 24F were SEM images of three kinds of mask materials, showing different viability of E. coli. Most E. coli on ACF and MBF maintained the integrity of the cell structure. The epifluorescence microscopy was used to differentiate the health conditions of E. coli (not shown). The results show prominent bactericidal ability of LIG, while little change of staining was observed on ACF and MBF.

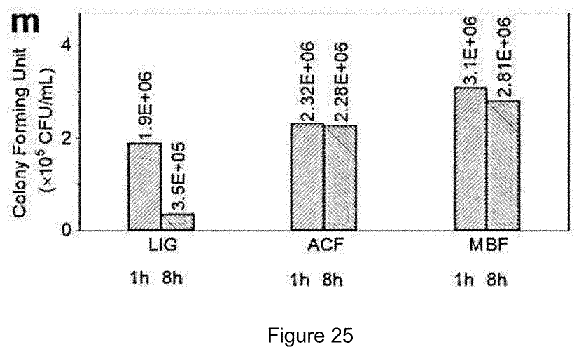

Colony forming unit (CFU) assay was further conducted to quantitatively compare the bactericidal efficiency. The optical images of the growth of E. coli on agar plate was shown in FIG. 25 was the statistics of CFU in different samples. Over 90% of the E. coli deposited on ACF and MBF remained alive even after 8 h. In comparison, the viability of E. coli on LIG dropped from 1.9×10⁶ CFU/mL to 3.5×10⁵ CFU/mL after 8 h (not illustrated).

The intrinsic antibacterial activity of LIG, ACF, and MBF has been found to be 81.57%, 2.00% and 9.13% respectively, which demonstrates advantageous safety of LIG over the commercial materials.

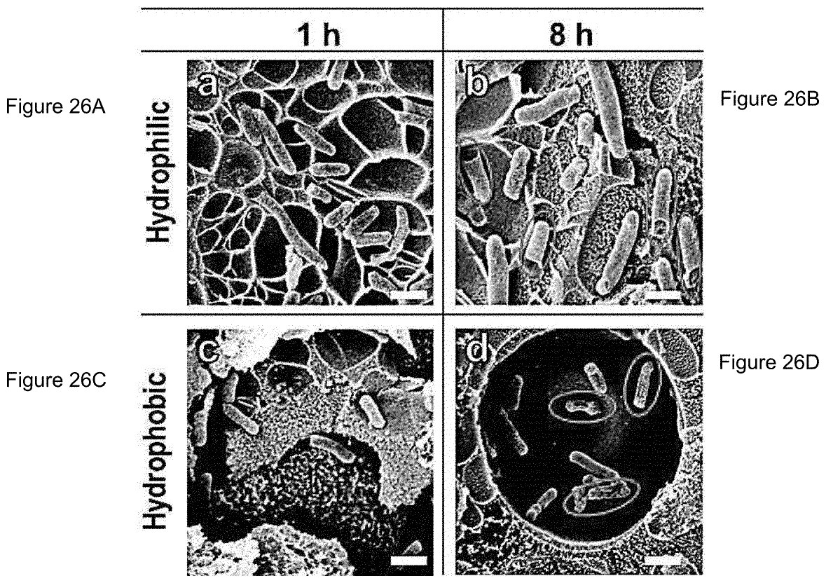

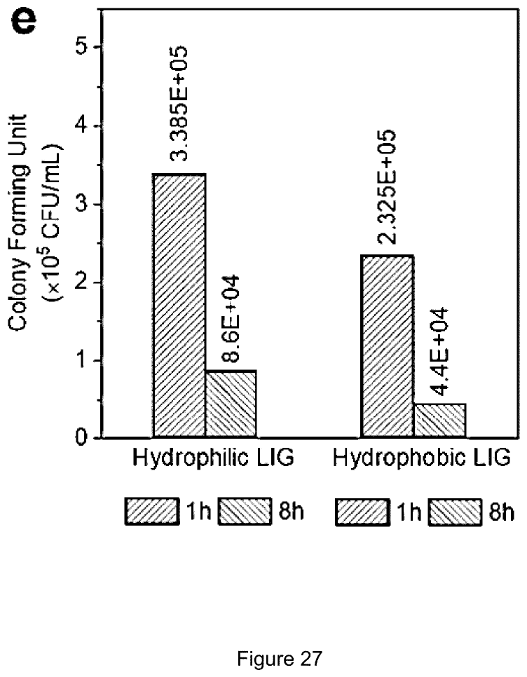

FIG. 26A to FIG. 26D shows the morphological difference of E. coli on hydrophilic LIG and hydrophobic LIG. As shown in FIG. 26A and FIG. 26C, the bacterial surface of control samples is round and smooth. In the experimental group, some of the E. coli cells on hydrophilic LIG (circled in FIG. 26B) are voided with disruption of outer membrane while that on hydrophobic LIG are shriveled and flattened (circled in FIG. 26D). Both are the typical morphology of bacteria losing the viability. The E. coli viability on two kinds of LIG was statistically summarized in FIG. 27. The bactericidal rate of hydrophobic LIG improves by about 7% when compared to hydrophilic LIG (74.5%). Both hydrophilic and hydrophobic LIG possess the moderate bactericidal ability.

The intrinsic bactericidal ability of LIG may stem from the irreversible damage induced by direct contact between bacteria and LIG. Also, rough surfaces, carbon nanofibers and micropores of LIG was reported to inhibit the attachment and proliferation of bacterial cells. Additionally, the interaction between sharp edge of graphene may also contribute to the bactericidal capacity of LIG. Due to the abundant oxygen-containing functional group such as —COON and —OH in hydrophilic LIG, the charge transfer between LIG and bacterial cell membranes may also cause the loss of intracellular substances.

For hydrophobic LIG, the induced dehydration is likely the main cause of death, as shown by the wizened shape of E. coli (FIG. 26D).

### Photothermal Effects of LIG in Expediting the Bacteria-Killing Efficacy

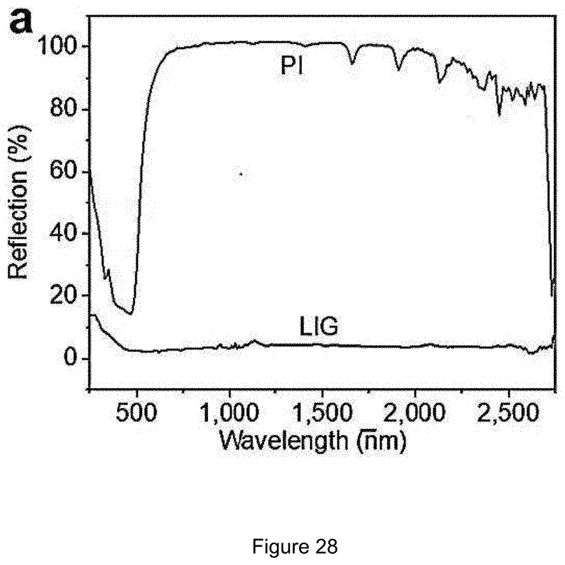

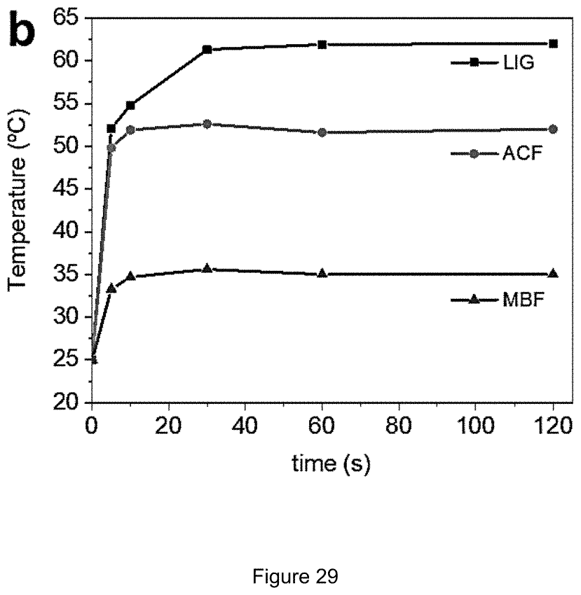

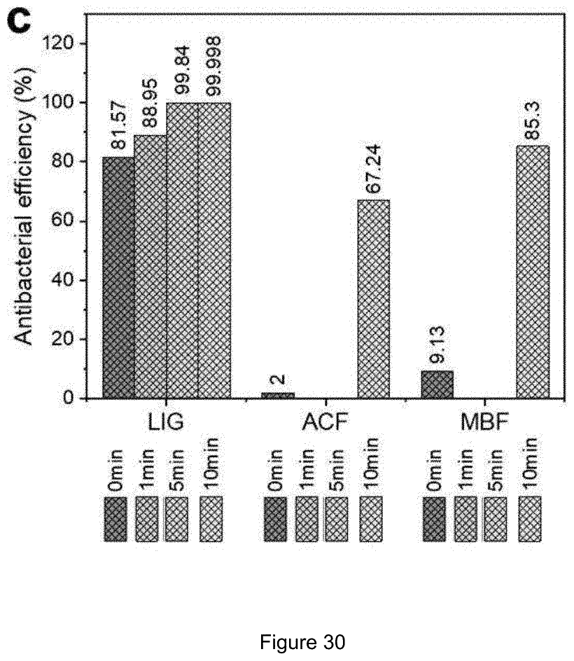

FIG. 28 shows the UV-Visible-Near Infrared spectrum of LIG and its precursor, which indicates that LIG can effectively absorb the energy from 250 nm to 2750 nm. FIG. 29 summarizes the change of temperature with time of LIG, ACF and MBF under 1 Sun irradiation. The surface temperature of LIG surged from 25° C. to 52° C. within 30 s and maintained at about 62° C. after further exposure to sunlight. The surface temperature of ACF was steady at about 52° C. after 30 seconds continuous irradiation, which is 10° C. lower than LIG. The lower temperature of ACF is due to the relatively large pores compared with LIG (Supplementary FIGS. 2c and 9a). The MBF was only 35° C. even after 60 seconds or longer irradiation. Then we used the CFU assay with different sunlight exposure time (1 min, 5 min and 10 min) to study the photothermal effect on bactericidal efficiency of LIG, ACF, and MBF. For all three kinds of materials, sunlight irradiation greatly enhanced the bactericidal rate, as shown in FIG. 30.

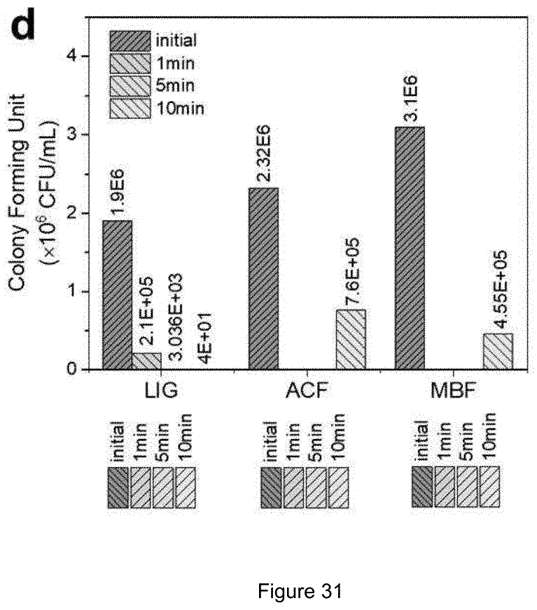

For example, the bactericidal capacity of ACF improved from 2% without sunlight to 67.24% with 10-min illumination. Similar enhancement was also observed for MBF with a germicidal capacity of 85.3%. The superior antibacterial efficiency of MBF over ACF may result from the hydrophobicity of MBF, which could accelerate the dehydration of E. coli upon exposure to sunlight. LIG showed remarkable bactericidal activity from the collective effect of intrinsic LIG properties and the photothermal enhancement. The bactericidal efficiency of LIG vastly improves to 99.84% and 99.998% after a 5-min and 10-min exposure to sunlight, respectively. It is worth mentioning that though ACF and MBF could kill over 65% and 85% of the bacteria after 10-min illumination, the amount of bacteria is still substantial. As shown in FIG. 31, the remaining number of viable E. coli on ACF and MBF was 7.6×10⁵ CFU/mL and 4.55×10⁵ CFU/mL after the photothermal treatment, respectively, while LIG only contained about 40 CFU/mL E. coli. Recent study suggested that the respiratory droplets and aerosols collected from individuals with acute respiratory symptoms for 30 min could contain 102×10⁵ copies of coronavirus or influenza virus5. Therefore, the moderate bactericidal activity of commercial masks might not warrant its safe use.

1. A facemask comprising:
a substrate having an inner surface for being worn over the mouth and nose of a person;
the substrate having an outer surface for facing away from the mouth and nose of the person; and
the inner surface comprises three dimensional graphene.

2. The facemask as claimed in claim 1, wherein
the three dimensional comprises laser induced graphene.

3. The facemask as claimed in claim 1, wherein
the three dimensional comprises hydrophilic graphene.

4. The facemask as claimed in claim 3, wherein
the graphene is more hydrophilic towards one side of the substrate and less hydrophilic towards the other side of the substrate.

5. The facemask as claimed in claim 4, further comprising:
a diode that is arranged across the graphene such that electrical potential difference generated in the graphene is able to light the diode.

6. The facemask as claimed in claim 4, further comprising:
electrochromic material that is arranged across the graphene such that electrical potential difference generated in the graphene is able to change the electrochromic material chromatically.

7. The facemask as claimed in claim 1, wherein
the substrate comprises any one of the following:
polyimide, paper, polyethersulfone, polysulfone, melt-blown fabrics, woven fabrics and felted-fabrics.

8. A method of functionalizing a carbonaceous material comprising the steps of:
providing a carbonaceous material;
a first stage of applying laser onto the carbonaceous material to produce a layer of three dimensional graphene in the presence of an inert atmosphere;
a second stage of applying laser onto the three dimensional graphene in the presence of air; wherein
the second stage comprises:
applying laser to a first part of the three dimensional graphene in such a manner that provides functionalization of the first part with polar groups; and
applying laser to a second part of the three dimensional graphene in such a manner that provides different extent of functionalization of the second part with polar groups.

9. The method of functionalizing a carbonaceous material as claimed in claim 7, wherein
functionalization of the first part with polar groups comprises applying a number of laser pulses to the first part; and
functionalization of the second part with polar groups comprises applying a different number of laser pulses to the second part.

10. The method of functionalizing a carbonaceous material as claimed in claim 7, wherein
functionalization of the first part with polar groups comprises applying one laser intensity to the first part; and
functionalization of the second part with polar groups comprises applying a different laser intensity to the second part.

11. The method of functionalizing a carbonaceous material as claimed in claim 7, wherein
functionalization of the first part with polar groups comprises applying one laser intensity to the first part; and
functionalization of the second part with polar groups comprises applying a different laser intensity to the second part.
